(19) United States
(12) Patent Application Publication
(10) Pub. No.: US 2005/0066968 A1
(43) Pub. Date: Mar. 31, 2005

(54) Generation, delivery, measurement and control of aerosol boli for diagnostics and treatments of the respiratory/pulmonary tract of a patient

(76) Inventors: Frederick M. Shofner, Knoxville, TN (US); F. Michael Shofner II, Knoxville, TN (US)

Correspondence Address: PITTS AND BRITTIAN P C, P O BOX 51295, KNOXVILLE, TN, 37950-1295, US

(21) Appl. No.: 10/499,473

(22) PCT Filed: Aug. 1, 2001
(86) PCT No.: PCT/US01/24183

Related U.S. Application Data
(60) Provisional application No. 60/222,575, filed on Aug. 1, 2000.
(60) Provisional application No. 60/222,273, filed on Jul. 31, 2000.
(60) Provisional application No. 60/251,114, filed on Dec. 4, 2000.

(30) Foreign Application Priority Data
Feb. 22, 2001 (WO) PCT/US01/05948

(51) Int. Cl. A61M 16/00

(52) U.S. Cl. 128/204.18

(57) ABSTRACT

In accordance with one aspect of the present invention for controlled delivery of medicament to the respiratory system of a patient, a measured quantity of a medicament, i.e., a bolus 32 is introduced into the inspiratory flow stream of a patient, via a breathing tube 18, and inhaled by the action of the patient's breathing cycle of the course of a breathing cycle of the patient, numerous properties or characteristics of the medicament, i.e., the bolus, the inspiratory flow, like properties or characteristics of the expiratory flow, and/or other useful information are derived. The derived information is employed to control subsequent delivery of boli to the patient, including the delivery of each bolus as a function of the commencement or progression of the breathing cycle, as input for calculations or determinations which are useful in analyzing the effectiveness of delivery of the medicament to the patient, patient compliance, direction of flow through the breathing tube, and other uses.

### RELATED APPLICATIONS

This application claims priority based upon pending U.S. Provisional Application Ser. No. 60/222,273, filed Aug. 1, 2000, entitled: METHOD AND APPARATUS FOR GENERATION AND INTRODUCTION OF AEROSOL BOLI INTO INSPIRATORY AND OTHER FLOWS, pending U.S. Provisional Application Ser. No. 60/222,575, filed Aug. 1, 2000, entitled: APPARATUS AND METHOD FOR MEASUREMENT OF AEROSOLIZED PARTICLES INHALED INTO AND EXHALED FROM THE PULMONARY SYSTEM; and pending U.S. Provisional Application Ser. No. 60/251,114, filed Dec. 4, 2000, entitled: METHOD FOR CONTROLLING PHARMACEUTICAL AND OTHER AEROSOL MASS DELIVERIES. Priority is also claimed based upon copending PCT application Serial No. PCT/US01/05948, filed Feb. 22, 2001, entitled MEASUREMENT OF AEROSOL MASS CONCENTRATION AND MASS DELIVERY RATE. Each of the forgoing copending applications is incorporated herein in its entirety by reference.

### FIELD OF THE INVENTION

This invention relates to the generation of aerosols and their delivery to and monitoring within respiratory and other flows for diagnostic and therapeutic purposes. The invention is more particularly directed to the generation of aerosol boli and their delivery under controlled conditions to a site within the respiratory system and more particularly to a pulmonary use site. Deliveries to other medical and non-medical use sites are also enabled by the present invention.

### BACKGROUND OF THE INVENTION

It is well known that a substantial fraction, perhaps 20%, of the world population suffers from respiratory ailments or dysfunctions. Asthma is a serious problem and is becoming more prevalent. Other ailments comprise common allergenic reactions or more serious medical conditions. Treatment of these ailments often involves the periodic (timed) administration of an aerosolized dose of a medicament to the patient via the patient's respiratory tract. By medicament is meant the active and inert, if any, components of a therapeutic drug prescribed by a physician on the basis of presented and/or diagnosed symptoms. Common inhalers are one example of this mode of introduction of aerosolized medicament to the respiratory tract of a patient.

Whereas the prior art methods and apparatus for treatment of diagnosed respiratory/pulmonary ailments or dysfunctions are useful, they suffer from problems associated with inadequate diagnoses, with inconsistent quantities of medicament being dosed, with inefficient deliveries to the diseased site(s), and the timing of the dosage relative to pulmonary inflow, and with patient compliance, among other concerns or problems. In certain instances, dosage can be critical in that (a) the treatment requires some minimum level of medicament for its effectiveness or (b) certain medicaments can be damaging or even lethal if administered in an excessive dosage. Of particular concern is the clinical laboratory setting in which a known quantity of a medicament desirably is inhaled by a patient repetitively and with known, meaning monitored and documented, accuracy and precision for diagnostic or therapeutic purposes. Of greater concern is non-supervised or self-treatment in a home setting; patient compliance and accuracy and precision of the proper drug to the proper site are major issues here.

Another background need arises from the rising preference for administration of certain drugs via the respiratory path, such as insulin, for example. In this case the therapeutic benefits of the medicament lie outside the respiratory tract, but their administration is preferably via the respiratory process. Improved methods and apparatus are essential for such treatments and are enabled by the present invention.

### SUMMARY OF INVENTION

In accordance with one aspect of the present invention, a measured quantity or dose of a medicament in the form of an aerosol bolus is generated by a bolus generator and introduced into the inspiratory flow stream of a patient. Such introductions may be at one or more times or volumes within an inspiratory portion of a breathing cycle, hence at one or more points in the inspiratory volume. Of those aerosolized particles which are delivered to the patient's respiratory tract all or a portion thereof may be deposited at a location or region within the respiratory tract, such as within the pulmonary system, and any non-deposited particles are exhaled by the action of the patient's expiratory breathing flow.

The present inventors have found that the identification of the deposition site of those aerosol particles which are deposited within the respiratory tract is strongly influenced by the timing of the bolus introduction into the inspiratory flow and by the size distribution of the aerosolized particles in the bolus.

In accordance with the present invention, in the course of a breathing cycle of the patient, numerous properties of the medicament, i.e., the aerosol bolus, and fluidynamic and other properties of the inspiratory and expiratory flows, and/or other useful information are measured and used for optimal control of subsequent dosages. The measured aerosol and air flow data are employed to control subsequent delivery of boli to the patient, including the type of medicament, dosage amount, particle size distribution, and the timing of the delivery of each bolus with respect to the inflow, among other things. Patient compliance and treatment regimen are stored for examination by medical personnel either in the clinical laboratory or remotely, over the internet, if, for example, treatment is in a home or small clinic setting. Treatment regimen may be supervised and/or altered locally or remotely.

The present invention is applicable and useful in both therapeutic treatment of a patient and in diagnosing an ailment. In a diagnostic embodiment of the present invention, challenge or diagnostic aerosols are generated and delivered to the patient's respiratory inflow and the fluidynamic properties and the aerosol properties are jointly monitored, as above for the therapeutic embodiment. Indeed, the apparatus is in many cases the same. In this diagnostic embodiment, the aerosol and air flow characteristics are carefully monitored and recorded, and characteristic waveforms similar to an electrocardiograph (EKG) or electroencephligraph (EEG) are presented. From these charts physicians may advantageously perform diagnoses of respiratory function, for example.

In an extension and/or combination of the therapeutic and diagnostics embodiments, automated, on-going diagnoses may be interspersed with therapeutics to optimize treatment according to a predetermined treatment regimen prescribed by the physician.

For present purposes, in a human respiratory environment, an aerosol bolus may be defined as a small volume of gas, ranging from microLiters to Liters, with typical bolus volume in the order of magnitude of approximately 1 milliliter, in which aerosols are added or “seeded” for subsequent transport and delivery. In most cases the aerosol sizes will correspond to inhalable or respirable particles, those typically in the range of 1 to about 10 um in aerodynamic equivalent diameter, but some cases require larger particles, notably upper respiratory tract diagnostics/treatments. Each such bolus is thus a localized, two or more component fluid, i.e., one or more components of gas, typically air, and one or more components of aerosols. Aerosols are liquid or solid particles which are generated from liquid, powder, or a combination of liquid and powder, feedstocks. Aerosol mass concentration is high centrally of the bolus and approaches zero toward its perimeter.

For illustrative purposes, aerosol boli, for introduction into inspiratory flows, may be further characterized as having mass concentrations of 1000 μg/L=1 g/m³, “respirable” particle size of order 1 μm diameter, and mass density=1.5 g/mL particles. The gas component of the bolus volume of 1.0 mL would normally be clean air, at standard temperature and pressure, and with 65% relative humidity. Further, within a bolus, the entrained particles may be monodisperse (i.e., the individual particles are of approximately the same diameter) or polydisperse (i.e., the individual particles exhibit a range of particle diameters).

It is to be noted that these introductory or inhaled aerosol characteristics are modified upon the aerosol (or a portion thereof) passing through the respiratory system and exhaled. These modifications are monitored in the expiratory flow. The more qualitatively evident of these modifications are losses within the respiratory system, particularly size-dependent losses, and changes in shape of the bolus concentration profile. Importantly, these modifications are different between normal and respiratory-compromised persons and for this, and other reasons, it is of importance to have a knowledge of these modifications for diagnostic and for therapeutic purposes, and/or for purposes of optimally controlling the characteristics of boli introduced into inspiratory flows.

Various parameters relating to the bolus and respiratory flows are monitored as the bolus passes from their source and into the breathing tube. In one embodiment, for example, the aerosol mass concentration and the aerosol particle size distribution are monitored as functions of time along with the inspiratory and expiratory volumetric flow rates. Representative electrical signals of each of these parameters are generated and fed to a controller, such as a microprocessor. Further, the direction of flow of the gaseous medium moving through the breathing tube is also monitored and an electrical signal representative of whether the flow is associated with inspiration or expiration by the patient is developed and fed to the controller.

Upon that expiration of the patient immediately following the inspiration of the bolus last inhaled, the flow of breath from the patient is detected and analyzed for parameters such as aerosol mass concentration, particle size distribution (taking into account particle growth in the nearly 100% RH environment of the lung), and volumetric flow, importantly, all as functions of time. Comparison of the data collected from monitoring the inflow of the aerosolized bolus and from monitoring that outflow of air from the patient upon that expiration action which first occurs after a given inhalation action, provides information on any of several results, including, for example, that quantity of the inhaled bolus which remained within the patient's respiratory tract during the observed breathing cycle. Notably, the present analysis is performed on a bolus by bolus basis, thereby providing valuable information on the efficiency of administration of the medicament and other information such as patient compliance. It may be appreciated that these and other data indicate the interactions of the aerosol bolus within the patient's pulmonary tract and, thereby, the performance thereof. It follows that the bolus generator characteristics may be controlled to optimize diagnostics or therapeutics.

Employing the electrical signals fed to the controller, the controller generates an electrical signal which is fed to one or more fast-acting solenoid valves, for example, which control the flow of pressurized fluid into a receptacle or “pocket” containing a measured quantity of the substance to be aerosolized, thereby controlling the timing of discharge of a bolus into the patient's respiratory system.

As desired, other parameters may be monitored, such as the temperature, pressure, relative humidity, etc. of the inspiratory and expiratory flow created by the patient's breathing action. And all such monitored parameters may be recorded and communicated for supervision and intervention, locally in a clinical setting, or remotely, over the internet, for home or small clinic settings.

### BRIEF DESCRIPTION OF THE FIGURES

FIG. 1 is a representation of one embodiment of the present invention and depicting its employment with a patient;

FIG. 2 is a schematic representation of an alternative embodiment for the delivery of individual boli to a breathing tube;

FIG. 3 is a top view, partly in section, of the turntable of FIG. 2;

FIG. 4 is a schematic representation of a further embodiment of the present invention and depicting the inhalation/exhalation of a bolus through the respiratory system of a patient;

FIG. 5 is a graph depicting the temporal behavior of bolus aerosol mass concentration and inspiratory and expiratory flows over time;

FIG. 6 is a graph depicting the mass of bolus deposited in the respiratory system during a breathing cycle of a patient;

FIG. 7 is a graph depicting an enlarged view of the expiratory aerosol mass concentration associated with a bolus introduced into the inspiratory flow of a patient at a given time and volume of inspiratory flow; and,

FIG. 8 is a graph depicting a specific treatment regimen depicting the profile of the mass concentration of medicament retained by a patient over time.

### DETAILED DESCRIPTION OF INVENTION

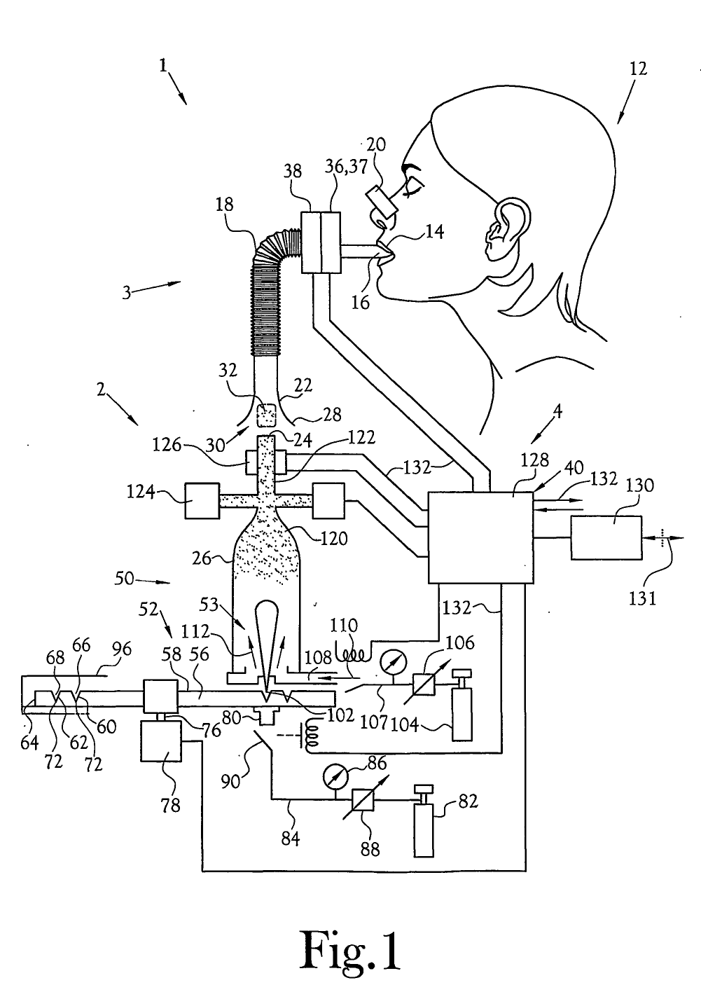

FIG. 1 depicts one embodiment of the present invention comprising an aerosol bolus diagnostic/therapeutic system 1 including a bolus generator 2, aerosol and gas flow monitoring sensors 3, and control and communications module 4. There is further depicted in FIG. 1 a patient 12 receiving within his/her mouth 14 a proximal end 16 of a flexible and instrumented breathing tube 18. Preferably, the nasal cavities of the patient are closed as by a clamp 20, thereby causing inhalation and exhalation by the patient via their mouth only.

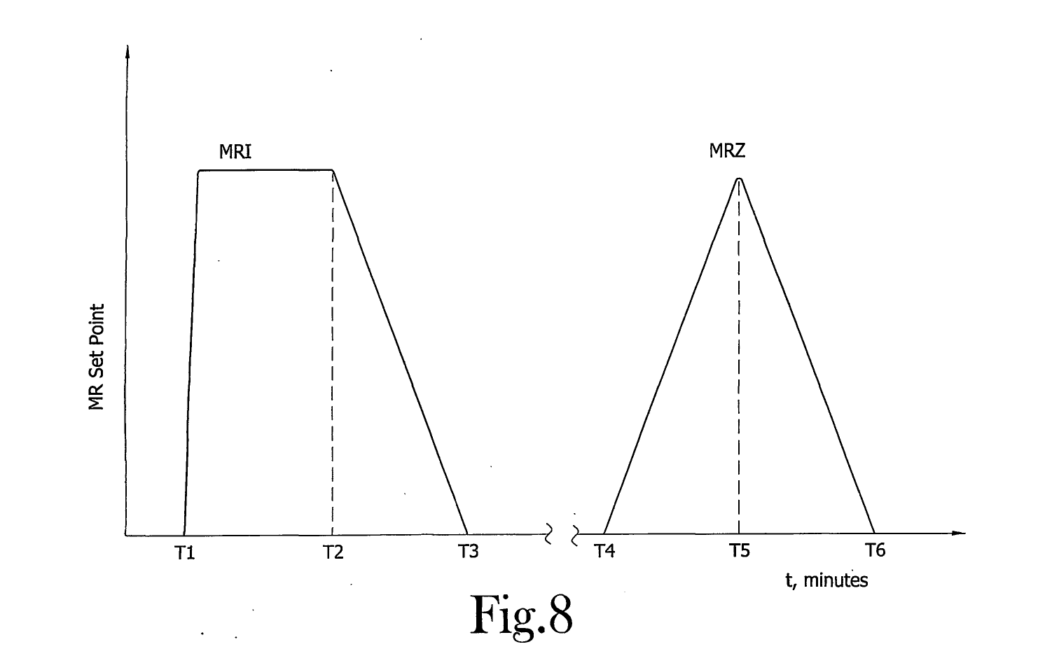

Referring to FIG. 1, as noted, generation and delivery of individual boli of aerosolized medicament, for example, is effected by a bolus generator 1, a first part of which is an aerosol generator 50. The aerosol generator 50 depicted in FIG. 1 includes a medicament delivery subassembly 52, aerosolizing section 53, and a mixing/stilling vessel 26 having an exit 24. Copending PCT application Serial No. PCT/US00/08354, filed Mar. 30, 2000, entitled: CONTROLLED DELIVERIES AND DEPOSITIONS OF PHARMACEUTICAL AND OTHER AEROSOLIZED MASSES, discloses apparatus for aerosolization and deposition of a measured quantity of a pharmaceutical onto a collector to develop a pill or like dose of the pharmaceutical which is subsequently intended to be ingested by a patient. This copending PCT application is incorporated herein in its entirety by reference and it is noted that the aerosol generator 50 of the instant application and aerosol generator 103 in FIG. 8 of the referenced and copending PCT application are substantially identical. However, neither this copending application, nor any other known prior art, discloses or anticipates the concept of controlled delivery of a measured bolus to a patient via the inspiratory portion of the patient's breathing cycle. Neither is there disclosed or anticipated in the known prior art the concept of monitoring the expiratory flow portion of a patient's breathing cycle and using that information to determine net delivery or medicament dosage to the patient. Further, all known prior art is silent on the matter of relating deliveries during inspiration to returned aerosols in the expiratory flow portion of the patient's breathing cycle as the basis for controlling, and/or selectively adjusting, the volume or mass concentration of each subsequent bolus which is delivered for inhalation by the patient as a function of the results observed with respect to each inhaled bolus.

The medicament delivery subassembly 52 of FIG. 1 includes a rotatable disc 56 having a substantially planar, flat, upper surface 58. Disposed in concentric paths, for example, which are disposed radially inwardly of the outer circumference 64 of the disc, there are provided a large plurality of individual receptacles, i.e, “pockets” 60 at spaced apart locations along their respective one of the paths. Each pocket is of a measured volume on the order of 1 cubic mm or, typically, smaller and opens outwardly of the upper surface 58 of the disc. There may be thousands of pockets 60 in tens of concentric circular patterns in a disc 56 having a diameter of about 300 mm. The bottom of each pocket 60 is provided with an air flow channel or small hole 72 through which compressed gas or air (or other fluid) may be fed in sufficient volume and flow rate as will dispel and aerosolize the contents of a given pocket in a direction normal to the plane of the upper surface 58 of disc 56.

The disc 56 is mounted for rotation about a vertical axis as defined by the vertical shaft 76 of a motor 78. The motor 78 is chosen to be of a stepper type which is capable of incremental rotational movement to thereby position one of the pockets 60 over and in registered operative relationship to a tube 80 that is in fluid communication between the flow channel 72 of a pocket and a source of pressurized air 82 as by a conduit 84. Interposed along the length of the conduit 84, there is provided a pressure regulator 88 and a pressure gauge 86 or electronic pressure sensor. A fast-acting solenoid valve 90 is interposed between the conduit 84 and the tube 80 for control over the timing and quantity of pressurized air to be admitted through the tube 80 and the flow channel 72 of a pocket disposed in registration with the tube 80. Power and control signals for actuation of the solenoid valve 90 are supplied from the controller 40. As desired, the turntable 56 may be enclosed to the extent desired as by a housing 96.

Referring yet to FIG. 1, when a given pocket 60 containing a measured quantity of medicament, for example, is disposed so that its bottom inlet hole 72 in register with the tube 80, the top of this same pocket 60 is disposed in register with the open bottom entry 102 of the mixing/stilling vessel 26. This registration of the pocket 60 with respect to the tube 80 and the open bottom entry 102 of the mixing/stilling vessel positions the medicament held within the pocket in line to be aerosolized by a short duration burst of pressurized gas delivered through the tube 80, into the bottom of the pocket 60, through hole 72, and thereby conveyed via hole 102 into the bottom of the mixing/stilling vessel 26. Seals and actuation of various sealing surfaces are known to those skilled in the art. It will be understood that the impulsive, aerosolizing gas flow may also be supplied from the top or sides of pocket 66.

Further, as seen in FIG. 1, additional pressurized gas from a source 104 thereof may be introduced through a pressure regulator 106 and through solenoid valve 107 into a channel 108 provided adjacent the entry end 102 of the mixing/stilling chamber 26. This “additional” pressurized air 110 may flow simultaneously with the flow of pressurized air through the pocket, or this additional flow may comprise a constant stream of air, or a combination of constant flow, interrupted by pulsed surges of pressurized air flow. This flow 110 may even draw gas from, rather than supply gas to, chamber 26, as its purpose is to optimally control the delivery of aerosol boli at the exit 24 of bolus generator 2. In any event, this additional air flow serves as another control variable to aid in the conveyance of the entrained medicament into the mixing/stilling vessel and/or to supplement or diminish the volume of air contained within the mixing/stilling vessel 26. In FIG. 1, the flow path of the additional air is depicted by the arrows 112 and generally constitutes an annular curtain of flowing air surrounding a conical-shaped volume of an expansive bolus 114 that is generated within the mixing/stilling vessel when the contents of a pocket are conveyed into the mixing/stilling vessel.

Within the mixing/stilling vessel 26, as noted, the highly energetic expansive bolus 114 effectively “explodes” into a conical geometry upon its entry into the mixing/stilling vessel. This action effectively disperses the medicament within the mixing/stilling vessel, while simultaneously permitting the entrained particles of the medicament to lose at least a major portion of their initial velocity, along with reduction in the velocity of the impulsively-supplied, aerosolizing gas via tube 80. This action comprises a “stilling” effect that tends to allow the particles of the medicament to “float”? within the interior volume of the mixing/stilling vessel and assume a substantially uniform distribution of aerosol mass concentration of such particles within the upper interior volume 120 of the mixing/stilling vessel, particularly near the exit 24 of the mixing/stilling vessel. It can thus be seen that the vessel 26 further serves as a reservoir, from which boli 32 are ejected.

Further, if the aerosol contains excessively large particles, or agglomerates, the mixing/stilling vessel also serves to classify, by vertical elutriation. That is, aerosols whose aerodynamic equivalent diameters exceed the size supported by the upward flow within the vessel will not ascend the column of gas defined in the interior of the vessel 26 and thus will be classified out of the effluent from the exit 24. The maximum size allowed to reach the output 24 is controlled by the average gas velocity inside chamber 26 which is in turn controlled by the cross sectional area and total gas volumetric flow rate, primarily by flows 110,112.

The exit end 24 from the mixing/stilling vessel is depicted in FIG. 1 as including a tapered top end 120 of the mixing/stilling vessel which directs flow from the vessel toward a central exit tube 122. A volumetric mass concentration and particle size distribution monitor 124 is disposed along the length of the exit tube 122 and provides an electrical signal or signals which are representative of the aerosolized state of the medicament particles disposed within the bolus 32 exiting the mixing/stilling vessel 26. The signals from sensor(s) 124 are fed to the controller 40. Suitable sensors 124 are available from ppm, Inc, Knoxville, Tenn. and are also described in copending application PCT/US01/05948. Also disposed along the length of the exit tube 122 there is provided a volumetric flow rate monitor 126 which develops an electrical signal which is representative of the volumetric flow through the exit tube. This electrical signal is also fed to the controller 40. These aerosol and flow monitors are used to control the bolus generator 2 itself. Further aerosol and sensor monitors 36,37,38, described later, are used to monitor and control the actual bolus 32 delivered to patient 12 upon inspiration and the modified bolus seen upon expiration.

The controller 40 may comprise internally one or more microprocessors, personal computers or programmable logic controllers (PLCS) 128, as all are well known in the art. Inputs to and outputs from controller 40 are fed to a stand-alone supervisory personal computer 130. If desired, outputs from the supervisory personal computer 130 may be fed to a “main frame” computer in the event additional processing capacity or speed of processing is needed or desired.

Within the controller 40, the various electrical signals are processed to provide multiple output signals. Some of these signals are employed to provide visual display of one or more of the data products generated by the individual monitoring elements of the present system, or of the results of calculations performed within the controller using the input signals from the monitor and/or other data, such as time between events, etc. It will be noted that the input/output (I/O) requirements of both the controller 40 and the personal computer 130 are extensive and robust performance is essential. The control features of the invention are further explained hereinafter.

In the present invention, it is desired that a prescribed and measured quantity, bolus by bolus, of a medicament be delivered by bolus generator 2 for subsequent delivery to a patient 12 for inhalation thereof during the inspiration portion of the patient's breathing cycle. Once the internal volume of the mixing/stilling vessel is substantially filled with aerosolized particles of the medicament, or once the aerosol mass concentration near the exit 24 of the bolus generator 2 approaches equilibrium, any subsequent introduction of gas into mixing/stilling chamber 26, whether additional medicament is introduced or not, will result in the expulsion of substantially equal volume of aerosolized medicament in a bolus from the exit 24 of the mixing/stilling vessel 26. It will be recognized that the rate of expulsion time of the stated volume of aerosolized medicament bolus 32. which may be on the order of hundreds of ms, will be a longer period of time than the time required to introduce the stated volume of medicament and pressurized air into the mixing/stilling vessel, e.g., tens of ms. This is due to the pressure differential between the internal volume of the mixing/stilling vessel relative to the atmosphere pressure. It will also be recognized that impulsive gas flow via solenoid valve 90 or controlling flows via solenoid valve 107 may be used to expel boli from exit 24, without moving disc 58. That is, more than one impulsive, expansive flow 114, without aerosols, may be delivered into the mixing/stilling vessel. Similarly, impulsive volumes of gas, without aerosolized medicament, may be effected by the auxiliary or additional flow 110.

Thus, the mixing/stilling vessel 26 operates to convert the initially violently turbulent inflow of pressurized gas and measured medicament particles in pockets 60 into the internal expansive bolus 114 to a relatively homogeneous concentration of aerosolized medicament particles in a bolus comprised of the aerosol-laden gas exiting exit 24. Accordingly, each such bolus 32 which exits the mixing/stilling vessel 24 comprises a volume which is controlled. Because of the mixing/stilling action within the vessel 26, the number and mass concentration of medicament particles within each exit bolus is controlled by the combined monitoring and controlling actions effected by aerosol monitor 124 and gas flow monitor 122. Thus, controller 40 is able to provide a measure of the quantity and other characteristics of the medicament in a bolus 32 exiting the bolus generator 2 and entering the breathing tube 18 and which is inhaled during the inspiratory portion of the breathing cycle of the patient 12.

It is noted that in one example, the pulsed fluid input to the mixing/stilling vessel results in a pulsed expulsion output of 1.0 mL of medicament and fluid, i.e., the bolus of interest, from the output of the mixing-stilling vessel. This bolus output occurs whether or not a fresh pocket containing additional medicament is presented for aerosolization because of the storage reservoir nature of the mixing/stilling vessel. That is, the gas volume entering the mixing/stilling vessel may be separately sourced from the impulsive, aerosolizing gas. The timing of the expulsion of a bolus from the mixing/stilling vessel and the quantity and other characteristics of aerosolized medicament contained within the bolus are thus functions of the timing and the quantity of the medicament and its associated entraining fluid entering the mixing/stilling vessel. It will be further recognized that the bolus exiting the mixing/stilling vessel is much lower in mass concentration and much slower in movement than the mass concentration of the medicament in the internal, expansive bolus entering the initial and internal parts of the mixing/stilling vessel. Expansive, aerosolizing boli 14, which are internal to the bolus generator 2 are to be sharply distinguished in their properties from external boli 32 intended for inhalation by a patient in accordance with the present invention.

In one embodiment, the exit from the mixing vessel is connected via a “soft” connection to a breathing tube, for example. In this embodiment, seen in FIG. 1, that end of the breathing tube 18 disposed adjacent the exit 24 from the mixing/stilling vessel is spaced apart from such exit by a relatively short distance to thereby define an annular opening 30 to the ambient atmosphere for the passage of exhaled air from a patient into the ambient atmosphere, while also defining a passageway for the movement therethrough of an expelled bolus 32, plus a portion of the ambient atmosphere, upon an inspiratory action by the patient. Since a typical inspiratory volume is about 1 L, it follows that the typical bolus volume of 1 mL is much smaller, so most of the inspiratory or expiratory volumes are through the annular region.

More specifically, the opposite and distal end 22 of the breathing tube 18 terminates adjacent, but spaced apart from, the exit 24 of a mixing/stilling vessel 26. In FIG. 1, the distal end of the tube is depicted schematically as comprising an outwardly flared inlet 28 that surrounds the exit 24 and defines therewith an annular passageway 30 leading from ambient atmosphere external of the tube and into the tube itself. This “soft” connection between the distal end 22 of the breathing tube 18 and the exit 24 of the mixing/stilling vessel 26 provides for alternate inspiration of ambient air and a bolus of medicament 32 and expiration of breath and bolus residuals from the patient. Expiratory flow from the patient may be blocked from entering the output 24 of the bolus generator 2 by diverter valves, if such isolation is required or desired.

Intermediate the opposite ends of the breathing tube 18 there are provided an aerosol monitor 36 and a flow direction and volumetric flow monitor 37,38 which respectively are capable of generating electrical signals that are representative of the aerosol characteristics, such as mass concentration and particle size distribution and electrical signals that are representative of the volumetric flow rate and direction of inspiratory and expiratory air to and from patient 12. Other fluidynamic data, such as pressure, humidity and the like, and patient data, such as breathing rate, heart rate, blood pressure, and the like, may also be monitored and recorded. Each of the electrical signals from the monitor 36, the flow direction detector and from the volumetric flow monitor are fed to the controller 40.

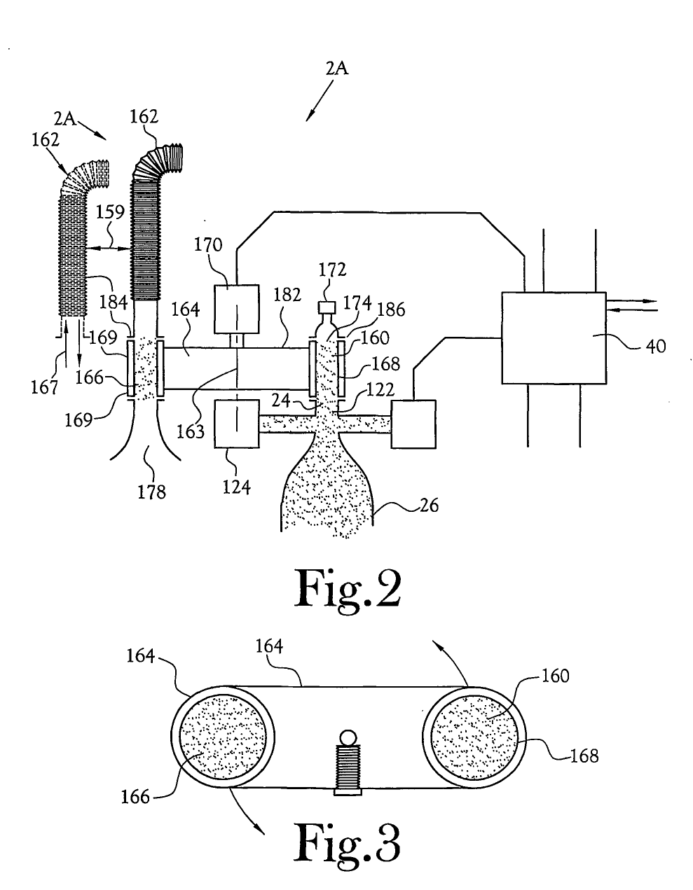

In FIGS. 2 and 3, there is depicted an alternative embodiment of the aerosol bolus generator subsystem 2A. This alternative replaces the “soft” connection of FIG. 1 with an apparatus and method providing more definitive bolus introduction, as well as better isolation of the bolus from ambient atmosphere, etc. In this alternative embodiment, the bolus generator 2 and aerosol and gas flow monitoring subsystems 3 are substantially the same as depicted in FIG. 1. Referring to FIGS. 2 and 3, there is depicted a subassembly 2A for the capture of a bolus 160 within a hollow capture and transport tube 168. When capture tube 168 is in registration with exit 24 of the mixing/stilling vessel, the bolus 160 is expelled from the mixing/stilling vessel 26 and loaded into tube 168. A previously loaded bolus 166 in transfer tube 169 is shown to the left, in registration with distal end 184 of breathing tube 162. In this position, bolus 166 is delivered to breathing tube 162. Immediately upon delivery, the breathing tube translates leftward, as indicated in FIG. 2 by the dashed lines and by motion arrow 159. In this leftmost position of the breathing tube, inspiratory and expiratory air flows move into and out of breathing tube 162, as indicated by flow arrows 165, 167.

More specifically, in the depicted embodiment, there is provided a rotary table 164 which rotates about a vertical axis 163 and has a plurality of cylindrical cavities 166,168 disposed concentrically of, and spaced inwardly of, the outer circumference 169 of the table 164. The table is mounted for rotation as by a motor 170 such that each of the cavities 166,168 are sequentially movable from a first position in register with the distal end 24 of the exit tube 122 to a second position in which the same cavity is in register with, hence in fluid flow communication with, the flexible breathing tube 162.

In operation of the subassembly 2A, when the cavity 168 is in register with the exit tube 122 of the mixing/stilling vessel, the other of the cavities 166 is in register with the breathing tube 162. When so positioned, the cavity 168 is available to receive therein a quantity of the aerosolized medicament contained within the mixing/stilling vessel upon the injection into the mixing/stilling vessel of a measured quantity of medicament, entrained in air, as further described hereinabove. A filter 172 is provided in fluid flow communication with that end 174, for example, of that cavity 168 which is in register with the exit tube 122 of the mixing/stilling vessel, to permit the expulsion of air from the cavity as the cavity is being filled with the bolus 160. Upon filling of the cavity 168 with the bolus 160, the table is rotated to exchange positions of the cavities 168 and 166, thereby moving the cavity 166 into register with the exit tube 122 and the cavity 168, which contains the bolus 160 in register with the breathing tube 18 and therefore available for inhalation by the patient. The distal end 178 of the breathing tube is open to ambient atmosphere to permit the patient to inhale a full breath of air and to sweep the bolus into the patient's pulmonary system.

As depicted and described hereinabove when discussing FIG. 1, the flow of the bolus through the exit tube 122 is monitored as by a detector 124 whose output is fed to the controller 40. In the depicted embodiment, the motor 170 is activated by a signal from the controller 40 and the change in positions of the cavities 166 and 168 may be completed in about 20 milliseconds. Appropriate housing for the table and the associated components is depicted schematically at 182 as are seals 184, 186, all as will be readily recognized by a person skilled in the art.

It will also be recognized that the practice of the invention necessitates attention to compactness of all the elements. Particular attention is paid to closely coupling the point of bolus introduction with the patient.

The disclosure hereinabove has focused on the controlled bolus generator subsystem 2, whose purpose is delivery of boli 32 of controlled characteristics for introduction in the respiratory tract of patient 12. The aerosol and gas flow monitoring subsystem 3 and the control and communications subsystem 4, together, monitor and control the actual aerosol mass and size distribution delivered to patient 12, upon inhalation, and received from patient 12, upon exhalation.

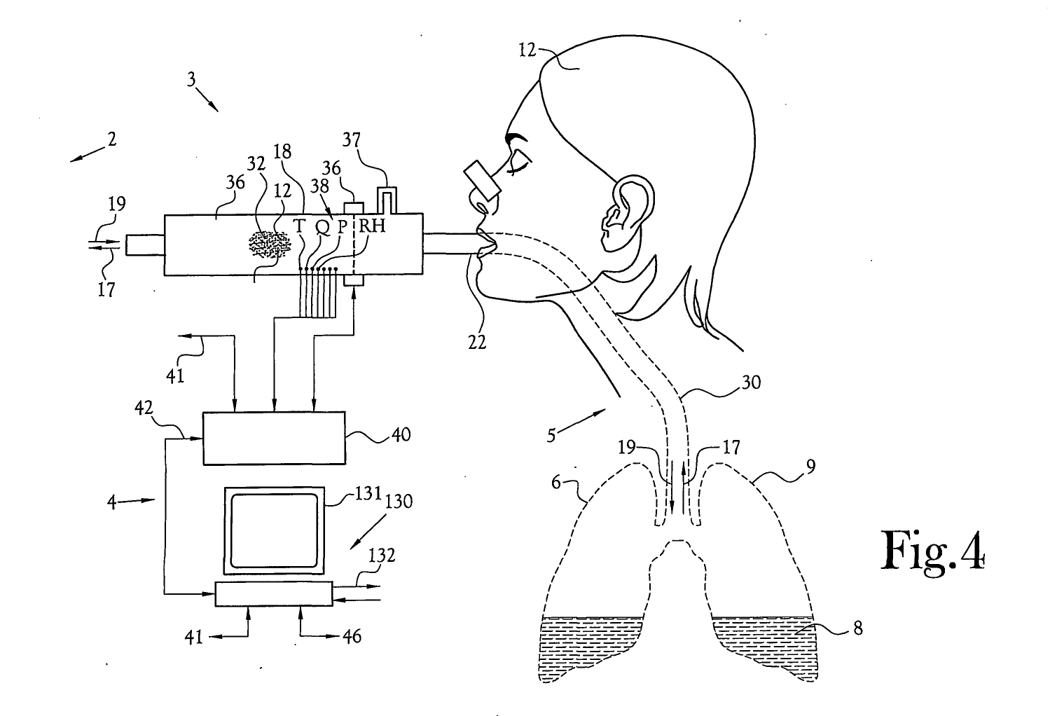

FIG. 4 depicts an aerosol bolus 32, after generation and introduction by bolus generator 1, being transported within breathing tube 18 during an inspiratory flow 19 by patient 12. The aerosol characteristics of bolus 32 and the fluidynamic properties of transporting gas flow 19 are monitored by aerosol sensors 36 and gas flow sensors 38, the latter of which are seen to consist of temperature, T, volumetric flow rate, Q, pressure, P and relative humidity, RH sensors, flow direction sensors, 37, and the like, and which are known in the art. Aerosol sensors 36 suitable for these purposes are described in copending application PCT/US01/05948 and are available from ppm, Inc, Knoxville, Tenn. Their function in the context of this invention is explained to the ends of establishing general performance criteria for others types of sensors that may be used in the invention.

The outputs of sensors 36,37,38 are connected, typically bidirectionally, to controller 40, which preferably is a dedicated micro controller-based system. Controller 40 also connects to bolus generator 2, and numerous other devices thereto related, for control of boli 32, as explained at the end of this disclosure. Data about the bolus and flow are sent from controller 40 to PC 130 which also receives other information relative to the diagnostic or therapeutic session in progress, some of it in essentially real time, via internet connections 44, internal main frame 46, and other controllers 132. Video screen 131 is the principle interface for in-clinic staff.

In a scalar analysis applicable to the present system, the rationale for which analysis is fully developed in copending PCT/US00/08354 and PCT/US01/05948, the mass delivery rate of aerosolized medicament to the inspiratory flow of the patient during each breathing cycle is determined by the formula:
dM/dT˜QC mg/sec Eq. 1
where M=mass delivered in g, Q=volumetric flow rate in liters/sec, and C=concentration of aerosolized medicament in g/liter. These values of Q and C are measured in instrumented breathing tube 18 by aerosol sensor 36 and flow sensor 38, ie, subsystem 3. A few comments are now directed to the aerosol sensor 36 since known separate sensors can provide suitable mass concentrations and size distributions and may be used in the invention. The purpose of these comments is to provide broad equivalent requirements for such separate aerosol sensors 36.

All known light scattering particle instrumentation sensors that are applicable to the present invention fall basically into two categories: photometers (or nephelometers) and single particle counters. Photometers respond to a plurality of particles in their scattering volume(s) at any given time and can give an indication proportional to mass concentration provided the particle characteristics are constant. That is, the calibration of photometers depends on particle size distribution, index of refraction, composition and shape of the particles being observed, transport gases, particle velocity and the like. Photometers cannot provide information on particle size. Single particle counters detect and size single particles in their scattering volumes. The strict requirement for no more than one particle in the scattering volume dictates a maximum particle concentration and limits the range in particle sizes that can be accommodated for a particular instrument design. It follows that single particle counters cannot provide mass concentration.

The electro-optical sensor described in the copending PCT patent application PCT/US01/05948 referenced hereinabove is a combination of both the photometer and the particle counter such that the primary data products are (a) accurate mass concentrations (g./L) and, (b) particle size distributions. Furthermore, these primary data products are provided for the inspiratory and expiratory flows and more particularly provide accurate, precise and cost-effective measurements of the mass concentration of relatively fine (mean diameter approximately 1 to 10 um) particles at very high concentrations (1-10,000 g./L) and over relatively short intervals of time (less than 1 ms to 10 s of ms). As a practical matter, making such measurements in view of real world variabilites in particle size distribution and concentration of medicaments intended to be administered via the respiratory tract of a patient and in view of the nearly 100% relative humidity environment encountered by the particles with the pulmonary tract is a difficult task. However difficult, a separate photometer in combination with a separate single particle counter may be used equivalently in the invention provided the performance requirements disclosed herein are met.

The 100% RH environment of the lung causes hygroscopic particles to grow. For certain diagnostic tests, hydrophobic particles are used. If the mass of hygroscopic particles is to be compared or balanced between inspiratory and expiratory flows, the growth must be corrected. Among the usual methods is to dry or desiccate them prior to measurement in the expiratory flow. This may be accomplished by the addition of a quick response heating element between the patient's mouth and the aerosol and flow sensor 36,38 station.

Aerosolized particles in the smaller particle size ranges (of order 1 μm) are primarily useful for accessing the deep alveolar regions of the lung 6 whereas those medicament particles in the larger range (about 10 um) deposit in the upper portion of the respiratory tract or conductive airways. In many diagnostic applications, nearly monodisperse particle size distributions are advantageous. In many therapeutic applications, polydisperse distributions, which are generally less complex, and therefore less expensive, to generate are advantageous. The present invention permits the use of such polydisperse medicaments in that the present invention provides for measurement of the particle size distribution of each bolus employed during each breathing cycle. Knowledge of the particle size distribution of a polydisperse bolus permits one to make comparisons and/or calculations which take into account the polydisperse nature of the particles of the bolus and thereby provide accurate and precise indication of the disposition of the particles within the pulmonary tract of the patient. This disposition includes the quantity of particles of a given particle size, hence their location of disposition within the respiratory system.

Referring to the FIGS. 1-4, attention is drawn to the respiratory tract 5 of patient 12 and particularly to lungs 6. Inspiratory and expiratory flows 19, 17, shown in trachea 30 and in breathing tube 18 are representative of the flow rates and directions. A bolus 32, upon transport into the respiratory system 5, interacts with the various parts of said system with the important result that the introduced bolus 32 is modified upon moving into and out of respiratory system 5. The types of modifications may readily be understood, qualitatively, as follows: losses of aerosols, because of impaction, Brownian motion, and settling, with loss rates dependent on particle size and local velocities, among other parameters affecting deposition; dispersion or broadening of the waveform, because of mixing and unequal flows due to unequal resistance and unequal retention times; skewing of the profile, for the same reasons; and increases in aerosol mass, for hygroscopic aerosols, because of growth in the nearly 100% RH environment inside the lungs 6. It is also qualitatively clear that the modifications to the original bolus concentration profile will be more pronounced for introductions that take bolus 32 to basal 8 versus apochal 9 regions of the lungs 6. It will be appreciated that these modifications will be quantitatively different between healthy persons and persons with respiratory dysfunction, for persons having the same physiological characteristics such as height, weight, age and the like. The diagnostic and the bolus generator 2 control features of the present invention accommodate the foregoing activities/environment associated with the act of administering a diagnostic or therapeutic aerosol via the respiratory tract of a patient.

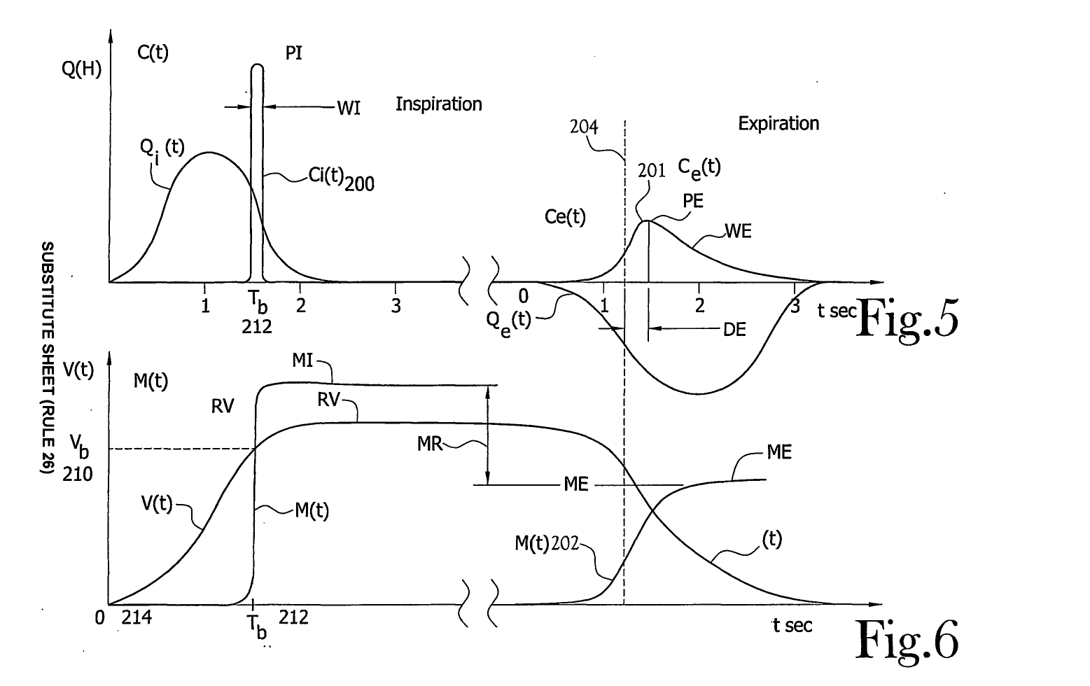

Modifications to the bolus, as quantitatively measured in expiratory flow 17, are rich in opportunities for diagnoses of respiratory health. FIG. 5 reveals the temporal behavior of bolus aerosol mass concentration C(t), as measured by aerosol sensor 36, and inspiratory and expiratory flows Q(t), as measured by flow sensors 38, FIG. 1. Note that inspiratory flow, positive Qi, is to the left and expiratory flow, negative Qe, is to the right of FIG. 5, respectively. It will be appreciated that the graphs of Q and C are of fundamentally different and distinct but time-related physical variables. FIG. 6 thus contains the same information but, to clarify disclosure, the time integral of Q(t)=V(t)=respired volume=RV at maximum is graphed instead of Q. Similarly, the integral of Ci(t)=M(t)=MI=Mass Inspired, maximum, for inspiration, and the integral of Ce(t)=M(t)=ME, at maximum, for expiration. Their difference, MI−ME=MR mass deposited in the respiratory tract is particular clear in FIG. 6. The time axes indicate typical times for inspiration and expiration.

Note further that the graph for inspired aerosol bolus concentration Ci(t) is a very short, “spiked,” waveform 200, compared to the graph for Qi(t). This is of course related to the small ratio between bolus 32 volume and respired volume, RV, as discussed above. This graph of Ci is characterized by its peak amplitude PI and by its full width at half maximum, WI, where I is associated with inspiration.

Significantly, it will be further appreciated that the qualitative modifications to the inspired bolus 32 waveform Ci 200 explained just above are confirmed in the concentration Ce(t) 201 waveform shown for expiration. Whereas FIG. 6 conveniently shows the mass deposited in the respiratory system MR, the qualitative and quantitative features of the expiratory aerosol concentration waveform Ce 201 in FIG. 5 are rich in diagnostic opportunities. We note here only the more elementary of such comparisons, noting first that the vertical dashed line 204 represents the ideal “return time” of the exhaled bolus, that is, with no or minimal modification, and for introduction at a given inspiratory volume Vb 210 and time, and Tb 212, FIG. 6. We note that inspiratory time is measured from the beginning, t=0 214 of inspiratory flow, as determined with flow rate and direction sensors 36, 37. Expiratory time is determined similarly. The most elementary features of expiratory concentration waveform Ce 201 are: peak amplitude PE, full width at half max WE, delay time DE, and skewness, which in this case is shown skewed right. (Mathematical forms for skewness are known.) E is associated with expiration.

But whereas the modified bolus waveform Ce 201 upon exhalation is evidently quite different from the inspired bolus Ci 200, it is comparisons of the Ces 201 for normal and compromised persons of similar physiological description that genuinely enable diagnostics. Further, comparisons based on bolus timing, as indicated by Vb 210 and Tb 212, on size distribution and type of diagnostic (or therapeutic) aerosols and the like enable thorough diagnostics and treatments. These comparisons between persons require, of course, that the test or diagnostic aerosols are the same, for a given test procedure or specific part of a given test procedure, in concentrations, size distributions, particle density and shape, and the like. Different test procedures will utilize different aerosols and other procedures, depending upon hypothesized ailment.

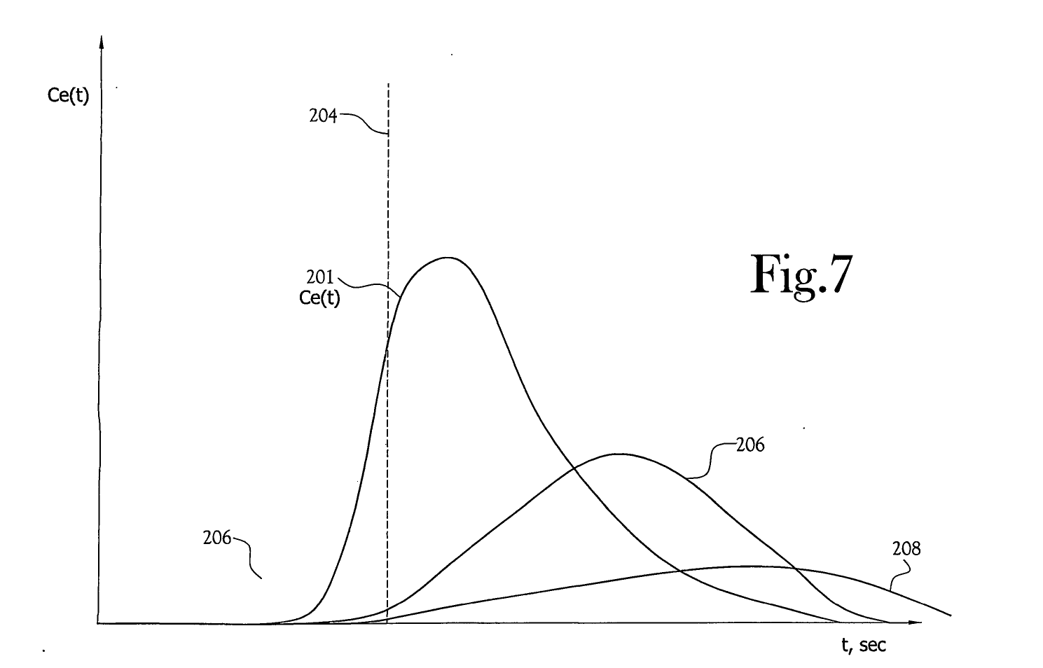

FIG. 7 shows an enlarged view of the expiratory aerosol mass concentration Ce(t) 201 associated with introduction of the inspiratory bolus 200 at an inspired test volume=Vb 210 and introduction time Tb 212, FIG. 6. Curve Ce 201, in either FIG. 6 or 7, is for members of a population of given physiological characteristic having normal respiratory performance. The waveform 206 is intended to be representative of persons of the same physiological characteristics with chronic upper respiratory tract obstructive disease and behavior, and waveform 208 is intended to be representative of persons with deep pulmonary disease, such as asthma.

Note that a test procedure embodying the present invention may involve a plurality of such tests, which tests as a battery would lead to respiratory performance profile. That is, a plurality of bolus injection timings, Tb 212 or Vb 210, over a range appropriate for the hypothesized ailment, would be executed and in combination would constitute a profile as contemplated in the present invention. One member of the set of waveforms is represented by bolus introduction Ci(t) 200, at Tb 212 or Vb 210 in FIG. 5 or 6, and leads to expiratory waveform Ce(t) 201 in FIG. 5. Such timings could correspond to basal use site 8, seen in FIG. 4. The plurality of these waveforms determines the profile which is used for diagnoses and to determine a treatment regimen. Execution of such tests is particularly aided by automatic controls of the bolus generator 2, for which controls are now disclosed.

Through the monitoring of the mass concentration and particle size distribution of medicament particles in bolus 32 with aerosol sensor 36 within the inspiratory and expiratory flows of breath from the patient, and through monitoring the volumetric flow rate with flow sensor 38 of the inspiratory and expiratory flows, the controller is capable of determining the mass of medicament or challenge aerosol inspired, MI FIG. 6, and the mass of exhaled aerosol, ME, and other characteristics of the aerosol inhaled into and exhaled from patient 12. Among other determinations, controller 40 is able to calculate the difference between the quantity and size distribution of medicament that is retained within the pulmonary tract of the patient per each breath taken. One such difference is the mass deposited in the respiratory system, MR, also FIG. 6. By monitoring and comparing the timing of the inspiratory and expiratory portions of the breathing cycle, along with the data relating to the amount of medicament retained in the respiratory tract of the patient, the controller is capable of providing an output which is indicative of the prescribed treatment regimen, as well as other valuable information. Timing above refers to either actual time from initiation of inspiratory flow, at which time bolus injection occurs (Tb 212) or the point in the inspiratory flow volume at which bolus injection occurs (Vb 210).

Another difference between inspired and expired boli is in particle size distribution. Since the deposition of aerosol mass in the respiratory tract is highly dependent upon particle size, it follows that changes in the size distribution between inspired and expired boli are also an indicator of respiratory performance. In some cases, size distribution changes are most sensitive and more robust indicators than total deposited mass MR.

The dosage to the respiratory tract MR may be controlled by controlling the mass introduced upon inspiration, MI, in at least two distinctly different ways, as follows. For example, if the controller 40 senses that the dosage MR has fallen relative to a set point, the controller 40 then increases the bolus 32 volume by increasing the amount of impulsively-supplied displacement gas via solenoid valve 107 until the desired MR is achieved. This is seen to be a fast control action which can operate on a individual bolus basis. Another control action, which is inherently slower, is to increase the equilibrium concentration at the top of mixing/stilling chamber 26, in the vicinity of the tapered section 120. This action is accomplished by increasing the rate at which fresh, filled pockets 66,68 of medicament are introduced to the aerosolizing section 53. The characteristic response time for this control action is related to the time for the equilibrium concentration in the vicinity of the output tapered section 120 to change. These two and other control strategies may of course be used in combination.

For some treatment regimens it is better to simply control the aerosol mass inspired, MI. The same fast, bolus volume and slower, aerosol concentration control actions, or others would be taken.

Of importance in the present invention is the ability of the apparatus to carry out the delivery of at least one bolus having the desired quantity of medicament therein to the patient during the inspiration portion of the patient's breathing cycle, to monitor the inflow and outflow during the breathing cycle, calculate any needed control signals and output such control signals (with or without changes) to the operative components of the present apparatus with the time span of a single breathing cycle of the patient. In general terms, a relaxed normal breathing cycle may consume between 1 and 5 seconds. Further, it will be recognized that two or more bolus generators may be employed to provide two or more boli, of different medicaments or different amounts of the same medicament, etc., to a breathing tube for inhalation by the patient. The later alternative will be recognized as being capable of delivering two boli per inhalation, alternate boli per alternate inhalations, or other similar combinations such as different timings of inhalation of the same medicament per each inhalation, alternation of diagnostic boli with therapeutic boli, etc.

FIG. 8 is a graph of a specific treatment regimen in which the medicament dosage set point MR follows the profiles shown. At time T1, the dosage quickly rises to MR1, which dosage is held constant by one or more control actions as described above. At time T2, dosage linearly decreases to zero at time T3. The interval from T2 to T3 may be of the order of 10 minutes. After a brief interval for change-over to another bolus generator 2 which contains a different diagnostic or therapeutic aerosol, the dosage set point linearly rises to maximum value MR2 and then linearly decreases to zero at T5. Change-overs are preferably automatic and can be accomplished in fractions of one second.

All of the above disclosure has been directed to diagnostics and treatments wherein the patient is able to breathe more or less normally. The invention of course applies to very ill patients for which inspiration and expiration are aided by a ventilator.

### INDUSTRIAL APPLICATION

The industrial applications of the present invention are as set forth herein above, along with other industrial applications which will be recognized by a person skilled in the art.

1. A method of administration of medicament to a patient in need thereof comprising the steps of
a) disposing a first quantity of medicament in a receptacle from which the medicament may be dispensed via pressurized gas,
b) injecting said quantity of medicament from said receptacle to the inlet of a mixing/stilling vessel having an inlet and an outlet spaced apart from said inlet employing pressurized gas flowing from a controllable source thereof into said receptacle containing the medicament, the volume and flow rate of said gas being sufficient to substantially entrain in said flowing gas the medicament contained in said receptacle, said vessel having an internal volume which is a multiple of the volume of the medicament and its associated volume of entrainment gas whereby the individual particles of the dispersed medicament are aerosolized as evidenced by their being substantially dispersed into individual particles of medicament and their velocity being reduced to substantially zero,
c) repeating steps a) and b) for a sufficient number of times as required to establish substantially equilibrium of said aerosolized medicament within said mixing/stilling vessel,
d) injecting a further measured quantity of medicament from a respective receptacle thereof or a further injection of pressurized gas into said inlet of said mixing/stilling chamber thereby expelling a bolus of aerosolized medicament from said outlet of said mixing/stilling vessel, said bolus being of a volume which is substantially equal in volume to the volume of said further measured quantity of medicament and its associated entrainment gas or said injected further pressurized gas
e) disposing the open distal end of a breathing tube in position to receive said expelled bolus,
f) by means of inspiration by the patient, drawing said bolus of said aerosolized medicament into the respiratory system of the patient,
g) intermediate said mixing/stilling chamber and the patient, generating a first electrical signal which is representative of the volumetric flow rate of aerosolized medicament entering the patient and generating a second electrical signal which is representative of the mass concentration of the aerosolized medicament entrained in the inspiratory flow of the patient aerosolized medicament exiting said mixing/stilling vessel,
h) feeding said first and second signals to a controller, wherein said first and second signals are employed to develop an output representative of the mass of the bolus delivered to the patient during inspiratory flow i) by means of exhalation by the patient, reversing the flow of fluid through said breathing tube,
j) detecting the direction of flow of fluid through said breathing tube,
k) upon detection of a change in flow of fluid through said breathing tube, generating a third electrical signal which is representative of the volumetric flow rate of fluid exhaled through said breathing tube, and generating a fourth electrical signal which representative of the mass concentration of aerosolized medicament contained within the fluid exhaled by the patient,
l) feeding said third and fourth electrical signals to said controller, wherein said third and fourth signals are employed to develop an output representative of the mass of said bolus exhaled from the patient during expiratory flow,
m) within said controller developing a further output which is representative of the difference between mass concentration of the bolus entering the inspiratory flow of the patient and the mass concentration of the bolus exhaled in the expiratory flow of the patient, and employing said difference, adjusting at least the injection of pressurized gas or quantities of aerosolized 75 medicament from respective ones of the receptacles thereof into said mixing/stilling vessel, hence at least the expulsion of boli from said mixing/stilling vessel, relative to the inspiratory portion of a breathing cycle of the patient.

2. The method of claim 1 wherein said first and second signals are employed within said controller to solve the equation:
$${Q}_{i} {C}_{i} = {\int }_{{d}_{t}}^{{d}_{m}} = {M}_{i} . \qquad Eq . 1$$

3. The method of claim 2 wherein the adjustment of the expulsion of a bolus is a function of the intake volume of the patient in the inspiratory portion of the breathing cycle of the patient.

4. The method of claim 2 wherein the adjustment of the expulsion of a bolus is a function of the time elapsed subsequent to the commencement of the inhalation portion of the breathing cycle of the patient.

5. A method for controlled delivery of a medicament to the respiratory and/or pulmonary system of a patient in need thereof including the steps of
a) aerosolizing the medicament,
b) dividing said aerosolized medicament into individual boli, each bolus comprising a known quantity of medicament,
c) by means of inhalation by the patient, delivering each bolus into the inspiratory flow of the patient, whereby the delivery of said boli is controlled in accordance with a predetermined regimen, and
d) measurement of the actual dosage of medicament delivered to the patient per bolus.

6. The method of claim 5 and including the steps of monitoring the volumetric flow rate of each bolus ejected from said mixing/stilling vessel and the mass concentration of medicament within said volumetric flow, and monitoring the volumetric flow of exhaled fluid from the patient and the mass concentration of medicament in said exhaled fluid, for each breathing cycle of the patient, and employing the values of said volumetric flows and mass concentrations to develop one or more control signals useful for adjusting the expulsion of a bolus into the inhalation stream of the patient.

7. The method of claim 6 wherein the timing of the expulsion of a bolus into the inhalation stream of the patient is a function of the time elapsed subsequent to the commencement of the inhalation portion of the breathing cycle of the patient.

8. The method of claim 4 wherein the adjustment of6 the expulsion of a bolus is a function of the intake volume of the patient in the inspiratory portion of the breathing cycle of the patient.

9. The method of claim 5 wherein the step of aerosolizing the medicament comprises injecting a measured quantity of medicament into a mixing/stilling vessel.

10. Apparatus for administration of medicament to a patient in need thereof comprising
a medicament delivery subassembly,
a mixing/stilling vessel in fluid communication with said medicament delivery subassembly and functioning to aerosolize medicament delivered to said vessel from said delivery subassembly, said aerosolized medicament being dispensed from said vessel in the form of a bolus,
a breathing tube having an open inlet end and being in fluid communication with a patient,
a first monitor disposed in operative proximity to said outlet from said mixing/stilling vessel and providing an output signal which is representative of the volumetric flow rate of aerosolized medicament expelled from said vessel,
a second monitor disposed in operative proximity to said outlet from said mixing/stilling vessel and providing an output signal which is representative of the mass concentration of aerosolized medicament expelled from said vessel,
a controller,
means conveying said signals from said first and second monitors to said controller,
a detector associated with said breathing tube and providing an output signal which is representative of the direction of flow of fluid through said breathing tube,
a further monitor associated with said breathing tube and providing output signals which are representative of the volumetric flow rate of fluid through said breathing tube during the inspiratory and expiratory portions, respectively, of a breathing cycle of the patient,
means conveying said signals from said detector and said third monitor to said controller,
means within said controller utilizing said signals from said first, second and third monitors and said detector to develop one or more output signals which are useful in selectively adjusting the expulsion of a bolus of aerosolized medicament from said mixing/stilling vessel for transfer thereof to said breathing tube.

11. The apparatus of claim 10 wherein the adjustment of the expulsion of a bolus is a function of the intake volume of the patient in the inspiratory portion of the breathing cycle of the patient.

12. The apparatus of claim 10 wherein the adjustment of the expulsion of a bolus is a function of the time elapsed subsequent to the commencement of the inhalation portion of the breathing cycle of the patient.

13. The apparatus of claim 10 wherein said medicament delivery subassembly includes a plurality of receptacles for like individual measured quantities of medicament selectively positionable relative to an inlet to said mixing/stilling vessel.

14. The apparatus of claim 10 and including a source of pressurized fluid in fluid communication with a receptacle which is in register with said inlet and a valve interposed between said source of pressurized fluid and said receptacle in operative position to deliver pressurized fluid into said receptacle to entrain and deliver to said mixing/stilling vessel said medicament disposed within said receptacle upon actuation of said valve.

15. The apparatus of claim 14 wherein said valve comprises a solenoid valve and signal transmission means from said controller to said valve, said valve being actuatable in response to a signal which is transmitted from said controller to said valve.

16. The apparatus of claim 10 wherein the internal volume of said mixing/stilling vessel is a multiple of the volume of entrained medicament and its associated entraining fluid injected into said vessel per each injection of medicament into said vessel.

17. The apparatus of claim 16 wherein said internal volume of said mixing/stilling vessel is on the order of 10 times the volume of medicament and its associated entraining fluid per each injection of medicament into said vessel.

18. The apparatus of claim 10 wherein said controller is either a microprocessor, a programmed logic controller, and/or a personal computer.

19. The apparatus of claim 10 wherein said mixing/stilling vessel includes an exit tube through which aerosolized medicament is expelled from said vessel and said first and second monitors are operatively associated with said exit tube.

20. The apparatus of claim 19 wherein said exit tube is disposed in fluid flow proximity to an open inlet end of said breathing tube for the receipt within said breathing tube of a bolus expelled from said exit tube.

21. The apparatus of claim 20 wherein said exit tube and said open inlet end of said breathing tube define an annular opening for the inflow of ambient atmosphere into said tube, along with said bolus, in the course of the inhalation portion of a breathing cycle of the patient, and for the outflow of fluid to the ambient atmosphere in the course of the exhalation portion of a breathing cycle of the patient.

22. The apparatus of claim 10 wherein the open inlet end of said breathing tube is disposed in laterally spaced apart relationship with respect to said mixing/stilling vessel and including a plurality of bolus receptacles mounted for sequential positioning between respective positions in fluid communication with said mixing/stilling vessel and positions in fluid communication with said open inlet end of said breathing tube, whereby at a given time one of said receptacles is disposed in position with respect to said mixing/stilling vessel to be loaded with a bolus and another of said receptacles is disposed in position with respect to said open inlet end of said breathing tube to deliver to said breathing tube a bolus contained within said another receptacle.

23. A method of controlled delivery of medicament to the respiratory system of a patient wherein maximization of deposition of medicament at a desired location within the respiratory system is achieved comprising the steps of
a) injecting a bolus of at least one diagnostic challenge aerosol into the inspiratory flow of the patient whereby the particles of said aerosol are entrained within said inspiratory flow and carried into the respiratory system of the patient;
b) monitoring the volumetric flow rate of the inspiratory flow of the patient within which the bolus is disposed;
c) determining the aerosol characteristics of the particles of said at least one challenge aerosol entrained within said inspiratory flow;
d) monitoring the volumetric flow rate of the expiratory flow of the patient following said inspiratory flow;
e) monitoring the aerosol characteristics of the particles of said at least one challenge aerosol entrained within said expiratory flow;
f) developing a profile of the difference between the particle size distribution of the aerosol particles in said inspiratory flow and the particle size distribution of the aerosol particles in said expiratory flow over the time of at least a single breathing cycle of the patient; and
g) selecting from said profile that point within the inspiratory flow which will result in maximum deposition/retention of aerosol particles within a given location within the respiratory system of the patient.

24. The method of claim 23 and including the step of injecting at least one bolus of medicament into the inspiratory flow of the patient at a time which will maximize the deposition/retention of medicament at a given location within the respiratory system of the patient.

25. The method of claim 23 and including the step of repeating steps a) through e) employing at least one further bolus of a challenge aerosol.

26. The method of claim 25 wherein the particle size distribution of said at least one further challenge aerosol is different from the particle size distribution of said at least one challenge aerosol.

27. The method of claim 23 wherein said aerosol characteristics comprise particle size distribution of the particles of the aerosol.
